(19) United States
(12) Patent Application Publication
(10) Pub. No.: US 2017/0258722 A1
(43) Pub. Date: Sep. 14, 2017

(54) ENCAPSULATING LIPOSOMES

(71) Applicant: COMFORT CARE FOR ANIMALS LLC, Middleton, WI (US)

(72) Inventors: Timothy D. Heath, Madison, WI (US); Lisa Ann Krugner-Higby, Belleville, WI (US); Lesley J. Smith, New Glarus, WI (US); Sheng Tu, Plymouth, MN (US); Natasha Kalkhof, Madison, WI (US); Rebekah Kay Franklin, Madison, WI (US)

(21) Appl. No.: 15/606,871

(22) Filed: May 26, 2017

Related U.S. Application Data
(63) Continuation of application No. 14/030,131, filed on Sep. 18, 2013.
(60) Provisional application No. 61/702,554, filed on Sep. 18, 2012.

(51) Int. Cl. A61K 9/127; A61K 47/02; A61K 31/485; A61K 31/65; A61K 31/4706; A61K 8/11

(52) U.S. Cl. CPC A61K 9/1277; A61K 31/4706; A61K 8/11; A61K 31/485; A61K 31/65; A61K 47/02; A61K 45/06

(57) ABSTRACT

Provided herein is technology relating to liposomes and particularly, but not exclusively, to compositions of liposomes encapsulating a biologically active agent, methods of preparing liposomes encapsulating a biologically active agent, and uses of liposomes encapsulating a biologically active agent to treat a subject.

### CROSS-REFERENCE TO RELATED APPLICATIONS

This application is a continuation of U.S. patent application Ser. No. 14/030,131, filed Sep. 18, 2013, which claims priority to U.S. Provisional Patent Application No. 61/702,554, filed Sep. 18, 2013, the contents of which are incorporated by reference in their entireties.

### STATEMENT REGARDING FEDERALLY SPONSORED RESEARCH OR DEVELOPMENT

This invention was made with government support under Grant No. 2 R01 RR 018802-05 awarded by the National Institutes of Health. The government has certain rights in the invention.

### FIELD OF TECHNOLOGY

Provided herein is technology relating to liposomes and particularly, but not exclusively, to compositions of liposomes encapsulating a biologically active agent, methods of preparing liposomes encapsulating a biologically active agent, and uses of liposomes encapsulating a biologically active agent to treat a subject.

### BACKGROUND

Liposomes, or lipid vesicles, are used for drug delivery to improve the therapeutic activity and increase the safety of a number of different pharmaceutical agents. Liposomal carrier systems (e.g., vesicles) are microscopic spheres of one or more lipid bilayers arranged around an aqueous core. The vesicles have been shown to be suitable as carriers for both hydrophilic and hydrophobic therapeutic agents owing to their unique combination of lipophilic and hydrophilic portions.

Liposomes are completely closed lipid bilayer membranes containing an entrapped aqueous volume. Liposomes may be unilamellar vesicles (possessing a single membrane bilayer) or multilameller vesicles (onion-like structures characterized by multiple membrane bilayers, each separated from the next by an aqueous layer). Liposomes may take other forms as well, e.g., multivesicular liposomes (MVL), which are lipid vesicles with multiple internal aqueous chambers formed by non-concentric layers and having internal membranes distributed as a network throughout the MVL. In these various forms, the bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

In a conventional liposome preparation such as that of Bangham et al. (J. Mol. Biol., 1965, 13:238-252), phospholipids were suspended in an organic solvent that was then evaporated to dryness to leave a phospholipid film on the reaction vessel. Next, an appropriate amount of aqueous phase was added, the mixture was allowed to “swell”, and the resulting MLVs were dispersed by mechanical means to produce multilamellar vesicles. This preparation provided the basis for the development of the small sonicated unilamellar vesicles described by Papahadjopoulos et al. (Biochim. Biophys, Acta., 1967, 135:624-638) and multilamellar vesicles.

Subsequently, techniques for producing large unilamellar vesicles (LUVs) such as reverse phase evaporation, infusion procedures, and detergent dilution were used to produce liposomes. A review of these and other methods for producing liposomes may be found in the text Liposomes, Marc Ostro, ed., Marcel Dekker, Inc., New York, 1983, Chapter 1. See also Szoka Jr. et al., (1980, Ann. Rev. Biophys. Bioeng., 9:467). One particular method for forming LUVs is described in Cullis et al., PCT Publication No. 87/00238, Jan. 16, 1986, entitled “Extrusion Technique for Producing Unilamellar Vesicles”.

Therapies employing bioactive agents can in many cases be improved by encapsulating the agent in liposomes rather than administering the free agent directly into the body. For example, incorporation of such agents in liposomes can change their activities, clearance rates, tissue distributions, and toxicities compared to direct administration. Liposomes themselves have been reported to have no significant toxicities in previous human clinical trials where they have been given intravenously. See, e.g., Richardson et al., (1979), Br. J. Cancer 40:35; Ryman et al., (1983) in “Targeting of Drugs” G. Gregoriadis, et al., eds. pp 235-248, Plenum, N.Y.; Gregoriadis G., (1981), Lancet 2:241, and Lopez-Berestein et al., (1985) J. Infect. Dis., 151:704. Liposomes are reported to concentrate predominantly in the reticuloendothelial organs lined by sinusoidal capillaries, i.e., liver, spleen, and bone marrow, and phagocytosed by the phagocytic cells present in these organs.

When liposomes are used in a liposome drug delivery system, a bioactive agent such as a drug is entrapped in the liposome and then administered to the patient to be treated. For example, see Rahman et al., U.S. Pat. No. 3,993,754; Sears, U.S. Pat. No. 4,145,410; Paphadjopoulos et al., U.S. Pat. No. 4,235,871; Schneider, U.S. Pat. No. 4,224,179; Lenk et al., U.S. Pat. No. 4,522,803; and Fountain et al., U.S. Pat. No. 4,588,578. Alternatively, if the bioactive agent is lipophilic, it may associate with the lipid bilayer. Typically, the term “entrapment” includes both the drug in the aqueous volume of the liposome as well as drug associated with the lipid bilayer.

Liposome formulations for pharmaceutical applications can be made either by combining drug and lipid before formation of the vesicles or by “loading” lipid vesicles with drug after they are formed. Upon administration to a patient, liposomes biodistribute and interact with cells in the body according to route of administration, vesicular composition, and vesicular size. Charge, chemistry, and bilayer components (e.g., the inclusion on the vesicle surface of protective polymers or targeting moieties) all change the way liposomes behave in the patient.

Mayer et al. found that the problems associated with efficient liposomal entrapment of the bioactive agents can be alleviated by employing transmembrane ion gradients (see PCT application 86/01102, published Feb. 27, 1986). Aside from inducing uptake, such transmembrane gradients also act to increase drug retention in the liposomes. For example, transmembrane pH gradients (4 pH) influence the drug loading of certain weak acids and weak bases. See, for example, Jacobs Quant. Biol. 8:30-39 (1940), Chapper, et al. in Regulation of Metabolic Processes in Mitochondria, Tager, et al. eds. Elsevier, Amsterdam, pp. 293-316 (1966), Crofts, J. Biol. Chem. 242:3352-3359 (1967), Crofts, Regulatory Functions of Biological Membranes, Jarnefelt, ed., Elsevier Publishing Co., Amsterdam, pp. 247-263 (1968), Rottenberg, Bioenergetics 7:61-74 (1975), and Rottenberg, Methods in Enzmol. 55:547-569 (1979). This behavior stems from the permeable nature of the neutral forms of these molecules, which contrasts with the impermeable nature of the charged forms. Thus, if a neutral amine (such as ammonia) diffuses across a biological membrane or vesicle exhibiting a ΔpH (e.g., with an acidic interior), it will become protonated and therefore become trapped in the vesicle interior.

Despite the earlier pioneering research in developing liposome formulations for pharmaceutical use, the further development of liposomes to administer pharmaceuticals has presented problems with regard to both drug encapsulation in the manufacturing process and drug release from the vesicle during therapy. For example, the use of liposomes to administer bioactive agents has raised problems with regard to both drug encapsulation and trapping efficiencies, and drug release during therapy. With regard to encapsulation, there has been a continuing need to increase trapping efficiencies so as to minimize the lipid load presented to the patient during therapy. In addition, high trapping efficiencies mean that only a small amount of drug is lost during the encapsulation process, an important advantage when dealing with the expensive drugs currently being used in some therapies. As to drug release, many agents have been found to be released rapidly from traditional liposomes after encapsulation. Such rapid release diminishes the beneficial effects of liposome encapsulation on efficacy and accelerates release of the drug into the circulation, causing toxicity, and thus, in general, is undesirable. Accordingly, there have been continuing efforts by workers in the art to find ways to increase entrapment efficiency and reduce the rate of release of bioagents and other drugs from liposomes.

Some conventional solutions to address these problems have used pH gradient loading (see, e.g., U.S. Pat. No. 5,837,282). However, in these approaches, the leakage of the drug from the liposomes in vitro was greater than for other types of liposomes (U.S. Pat. No. 5,837,282; Fenske, et al. “Ionophore-mediated uptake of ciprofloxacin and vincristine into large unilamellar vesicles exhibiting transmembrane ion gradients” Biochim Biophys Acta (1998) 1414: 188).

### SUMMARY

Better results for leakage have been achieved using ammonium sulfate gradient loading (see, e.g., FIG. 1), but the percentage of drug loaded is related to the concentration of ammonium sulfate. This has been shown in loading experiments in which increasing the concentration of ammonium sulfate in the liposome from 240 mM up to 1.5 M increased loading from around 35% to 67%. The leakage from liposomes loaded using 240 mM ammonium sulfate, wherein the hydromorphone is present in the liposomes as the sulfate salt, is less in vitro than for liposomes loaded with hydromorphone hydrochloride by passive aqueous capture (FIG. 1). Leakage of hydromorphone from liposomes loaded with different concentrations of ammonium sulfate, however, was not diminished by increasing the concentration of ammonium sulfate inside the liposomes (FIG. 2). These data suggest that leakage is related to the counter ion (e.g., sulfates are held in the liposome better than chlorides) and the pH in the liposome.

Accordingly, the technology provided herein maximizes both the use of a sulfate counter ion and provides a lower pH inside the liposome than the pH outside the liposome. In particular, sulfuric acid (instead of ammonium sulfate) is loaded directly into liposomes. As an exemplary application, loading opioid drugs under these conditions increases the concentration of drug loaded in the liposome relative to both directly-loaded liposomes and ammonium sulfate gradient loading, and decreases leakage even more than ammonium sulfate. As shown in the examples, liposomes were loaded with hydromorphone, chloroquine, and/or buprenorphine using the acid loading method and tests of encapsulation and leaking validated the technology.

Provided herein is technology relating to liposomes and particularly, but not exclusively, to compositions of liposomes encapsulating a biologically active agent, methods of preparing liposomes encapsulating a biologically active agent, and uses of liposomes encapsulating a biologically active agent to treat a subject.

Accordingly, in one aspect, the technology relates to a composition comprising liposomes, sulfate ions, and hydrogen ions, wherein the concentration of the hydrogen ions inside the liposomes (i.e., in the interior phase) is greater than the concentration of the hydrogen ions outside the liposomes (i.e., in the exterior phase). In some embodiments, the liposome compositions comprise an interior phase (i.e., the area inside the liposomes in the compositions) and an exterior phase (i.e., the area outside of the liposomes in the composition) which may preferably be an aqueous phase. In some embodiments, the liposomes compositions may be further described as a composition or system comprising an aqueous medium having dispersed therein liposomes encapsulating an intraliposomal aqueous compartment. In some embodiments, the compositions according to the technology comprise sulfuric acid. In some embodiments, the interior (i.e., the interior phase or intraliposomal compartment) of the liposomes has a pH of at least 3 pH units lower than the exterior of the liposomes (i.e., the external phase or aqueous medium in which the liposomes are dispersed). In some embodiments, the compositions comprise a bioactive agent in the interior of the liposomes. For example, in some embodiments, the compositions comprise an analgesic in the interior of the liposomes, e.g., an opioid, e.g., hydromorphone and/or buprenorphine. In some embodiments, the compositions comprise chloroquine. In some embodiments, the compositions comprise doxycycline. Some embodiments relate to compositions comprising such bioactive agents including, but not limited to, an antibiotic, an antitumor agent, an anaesthetic, an analgesic, an antimicrobial agent, a hormone, an antiasthmatic agent, a cardiac glycoside, an antihypertensive, a vaccine, an antiarrhythmic, an immunomodulator, a steroid, a monoclonal antibody, a neurotransmitter, a radionuclide, a radio contrast agent, a nucleic acid, a protein, a herbicide, a pesticide, and suitable combinations thereof.

In some embodiments, acid outside the liposomes is neutralized partially or wholly, e.g., with a base. Thus, in some embodiments, the compositions comprise a salt outside the liposomes, for example, sodium sulfate. The technology is not limited in the lipids from which the liposomes are produced. For example, in some embodiments, the liposomes comprise phosphatidylcholine. In some embodiments, the liposomes comprise a phosphatidylcholine selected from the group consisting of distearoylphosphatidylcholine, hydrogenated soy phosphatidylcholine, hydrogenated egg phosphatidylcholine, dipalmitoylphosphatidylcholine, dimyristoylphosphatidylcholine, and dielaidoylphosphatidylcholine. In some embodiments, the liposomes comprise sphingomyelin, neutral lipids (e.g., Niosomes), or acidic phospholipids. In some embodiments, the liposomes comprise dipalmitoylphosphatidylcholine and/or cholesterol. In addition, in some embodiments the liposomes are used in a pharmaceutical formulation. Accordingly, in some embodiments of the technology the compositions of liposomes further comprise an excipient and/or a pharmaceutically acceptable carrier.

The technology provides liposome compositions that efficiently encapsulate, and thereafter retain, a bioactive agent. Upon incubating the liposomes produced according to the technology with the bioactive agent, the bioactive agent moves into the interior spaces of the liposomes. Thus, in some embodiments, the bioactive agent in the interior of the liposomes is at least about 50%, at least 60%, or at least 70% of the amount of the bioactive agent that is added to the composition and incubated with the liposomes. In some embodiments, the bioactive agent in the interior of the liposomes is at least about 90-100%% of an amount of the bioactive agent added to the composition. In some embodiments, the bioactive agent is present at an amount of about 0.1 to 20 mg/μM phospholipid in the liposomes. In addition, after encapsulation, the liposomes retain the bioactive agent such that, according to embodiments of the technology, the compositions retain more than 50%, 60%, 70%, 80%, 90% or 95% of the bioactive agent in the liposome interior, e.g., for at least 72 hours.

The technology also relates to embodiments of methods for preparing liposomes encapsulating a bioactive agent, the methods comprising, e.g., forming liposomes comprising a concentration of hydrogen ions inside the liposomes that is greater than the concentration of the hydrogen ions outside the liposomes; and loading the liposomes with a bioactive agent by incubating the liposomes with the bioactive agent. In some embodiments, the interior of the liposomes has a pH of at least 3 pH units lower than the exterior of the liposomes. For example, in some embodiments forming the liposomes comprises forming liposomes in the presence of sulfuric acid. In some embodiments, acid outside the liposomes is partially or wholly neutralized by adding a base to increase the pH outside the liposomes. In some embodiments of the methods, the liposomes and bioactive agent are incubated at about 80° C. for more than about 1.5 hour. In some embodiments, after the liposomes are loaded, unencapsulated bioactive agent is removed, e.g., by washing the loaded liposomes. Accordingly, in some embodiments the methods further comprise washing the liposomes to remove unencapsulated bioactive agent. In some embodiments, the methods further comprise centrifuging the liposomes to remove unencapsulated bioactive agent.

The methods provided are not limited in the bioactive agent that is loaded in the liposomes. For example, in some embodiments, the bioactive agent is an analgesic, e.g., an opioid, e.g., hydromorphone and/or buprenorphine. In some embodiments, the bioactive agent is chloroquine. In some embodiments, the bioactive agent is an antibiotic, e.g., doxycycline.

In some embodiments, the technology relates to a method of loading a bioactive agent into liposomes, the method comprising contacting the liposomes with a solution comprising the bioactive agent, wherein the liposomes comprise a concentration of hydrogen ions inside the liposomes that is greater than the concentration of the hydrogen ions outside the liposomes. In some embodiments of the methods, the methods further comprise terminating the incubation by removing unencapsulated bioactive agent and isolating the liposomes comprising the encapsulated bioactive agent. In addition, the technology encompasses liposome compositions obtainable by any embodiment of the methods described herein in accordance with the technology.

According to the technology, provided herein are embodiments a liposome composition obtainable by a method comprising the steps of forming liposomes comprising a concentration of hydrogen ions inside the liposomes that is greater than the concentration of the hydrogen ions outside the liposomes; and loading the liposomes with a bioactive agent by incubating the liposomes with the bioactive agent. In some embodiments, the methods further comprise terminating the incubation by removing unencapsulated bioactive agent and isolating the liposomes comprising the encapsulated bioactive agent. In addition, the technology encompasses liposome compositions obtainable by any embodiment of the methods described herein in accordance with the technology.

The technology provides embodiments of a method of manufacturing liposomes that encapsulate a bioactive agent, the method comprising the steps of forming liposomes comprising a concentration of hydrogen ions inside the liposomes that is greater than the concentration of the hydrogen ions outside the liposomes; and loading the liposomes with a bioactive agent by incubating the liposomes with the bioactive agent. In addition, the technology relates to embodiments of pharmaceutical compositions comprising a composition according to the technology provided.

In some embodiments, the technology provides a composition comprising liposomes, sulfate ions, hydrogen ions, and a bioactive agent for use as a medicament, wherein the concentration of the hydrogen ions inside the liposomes is greater than the concentration of the hydrogen ions outside the liposomes. In some embodiments, the interior of the liposomes has a pH of at least 3 pH units lower than the exterior of the liposomes. Furthermore, in some embodiments, the technology provides a composition comprising liposomes, sulfate ions, hydrogen ions, and a bioactive agent (e.g., an analgesic or opioid) for use as a medicament to reduce pain in a subject, wherein the concentration of the hydrogen ions inside the liposomes is greater than the concentration of the hydrogen ions outside the liposomes. In some embodiments, the technology provides a composition comprising liposomes, sulfate ions, hydrogen ions, and a bioactive agent (e.g., an antibiotic) for use as a medicament to treat or assist in preventing infection in a subject, wherein the concentration of the hydrogen ions inside the liposomes is greater than the concentration of the hydrogen ions outside the liposomes.

In some embodiments of the compositions, the bioactive agent is an analgesic, e.g., an opioid, e.g., hydromorphone and/or buprenorphine and in some embodiments the bioactive agent is chloroquine. In some embodiments, the bioactive agent is an antibiotic, e.g., doxyclycline. In some embodiments of compositions, the compositions are obtainable by a method comprising the steps of forming liposomes comprising a concentration of hydrogen ions inside the liposomes that is greater than the concentration of the hydrogen ions outside the liposomes; and loading the liposomes with a bioactive agent by incubating the liposomes with the bioactive agent.

The technology is related, in some embodiments, to methods of treating a subject in need of pain reduction, the method comprising administering to the subject a composition according to the technology provided herein; and assessing the subject's pain. In some embodiments, the assessing is performed before the administering and in some embodiments the assessing is performed after the administering. In some embodiments, the assessing is performed both before and after the administering, and in some embodiments subsequent administering and/or assessing steps are performed. In some embodiments, the administering is changed, modified, and/or adjusted based on information attained during the assessing step. Thus, in some embodiments, the methods comprise assessing the subject's pain prior to the administering and in some embodiments the methods further comprise a second administering after the assessing.

The technology is related, in some embodiments, to methods of treating or assisting in the prevention of an infection, the method comprising administering to the subject a composition comprising an antibiotic according to the technology provided herein.

The methods are not limited in the types or classes of subjects to which the compositions are administered. For example, in some embodiments the subject is a mammal. In some embodiments, the subject is not a human subject. In some embodiments, the subject is under the care of a veterinarian.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a plot showing in vitro release of hydromorphone from freeze-thaw liposomes (diamonds) and ammonium sulfate gradient-loaded liposomes (squares).

FIG. 2 is a plot showing in vitro leakage of hydromorphone from liposomes prepared using different concentrations of ammonium sulfate.

FIG. 3 is a plot showing leakage of hydromorphone in vitro from liposomes prepared by the acid loading technology provided herein.

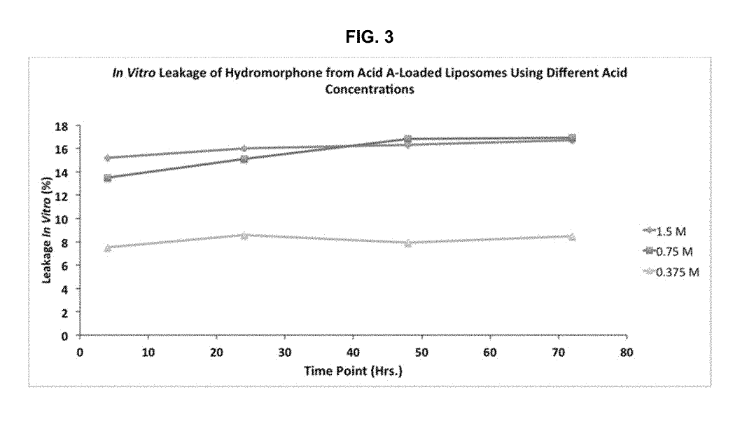

FIG. 4 is a plot showing leakage of buprenorphine in vitro from liposomes prepared by the acid loading technology provided herein.

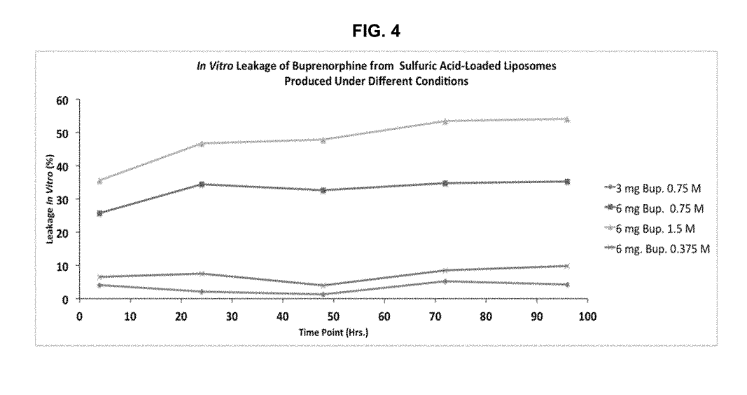

FIG. 5 is a plot showing in vitro leakage of doxycycline from DPPC liposomes made using different molar concentrations of sulfuric acid.

FIG. 6 is a plot showing pharmacokinetics of three preparations of doxycycline in rats.

FIG. 7 is a plot showing in vitro leakage of hydromorphone from DPPC liposomes made using two different molar concentrations of nitric acid.

FIG. 8 is a plot showing LE-Bup pharmacokinetics in rats administered a single dose of 3 mg/kg subcutaneously.

It is to be understood that the figures are not necessarily drawn to scale, nor are the objects in the figures necessarily drawn to scale in relationship to one another. The figures are depictions that are intended to bring clarity and understanding to various embodiments of compositions and methods disclosed herein. It should be appreciated that the drawings are not intended to limit the scope of the present teachings in any way.

### DETAILED DESCRIPTION

Provided herein is technology relating to liposomes and particularly, but not exclusively, to compositions of liposomes encapsulating a biologically active agent, methods of preparing liposomes encapsulating a biologically active agent, and uses of liposomes encapsulating a biologically active agent to treat a subject.

The section headings used herein are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the technology may be readily combined, without departing from the scope or spirit of the technology.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The term “lipid” refers to any suitable material resulting in a bilayer such that the hydrophobic portion of the lipid material orients toward the bilayer interior while the hydrophilic portion orients toward the aqueous phase. Hydrophilic characteristics derive from the presence of phosphato, carboxylic, sulfato, amino, sulfhydryl, nitro, and other like groups. Hydrophobicity could be conferred by the inclusion of groups that include, but are not limited to, long chain saturated and unsaturated aliphatic hydrocarbon groups and such groups substituted by one or more aromatic, cycloaliphatic or heterocyclic group(s).

Amphipathic lipids often find use as the primary lipid vesicle structural element. Examples of amphipathic compounds are phosphoglycerides and sphingolipids, representative examples of which include phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid and glycosphingolipid families are also within the group designated as lipid. Additionally, the amphipathic lipids described above may be mixed with other lipids including triacyglycerols and sterols.

“Phospholipid” refers to any one phospholipid or combination of phospholipids capable of forming liposomes. Phosphatidylcholines (PC), including those obtained from egg, soy beans, or other plant sources or those that are partially or wholly synthetic, or of variable lipid chain length and unsaturation find use in embodiments of the present technology. Synthetic, semisynthetic, and natural product phosphatidylcholines including, but not limited to, distearoylphosphatidylcholine (DSPC), hydrogenated soy phosphatidylcholine (HSPC), soy phosphatidylcholine (soy PC), egg phosphatidylcholine (egg PC), hydrogenated egg phosphatidylcholine (HEPC), dipalmitoylphosphatidylcholine (DPPC), and dimyristoylphosphatidylcholine (DMPC) are suitable phosphatidylcholines for use in this technology. All of these phospholipids are commercially available.

Further, phosphatidylglycerols (PG) and phosphatic acid (PA) are also suitable phospholipids for use in the present technology and include, but are not limited to, dimyristoylphosphatidylglycerol (DMPG), dilaurylphosphatidylglycerol (DLPG), dipalmitoylphosphatidylglycerol (DPPG), distearoylphosphatidylglycerol (DSPG) dimyristoylphosphatidic acid (DMPA), distearoylphosphatidic acid (DSPA), dilaurylphosphatidic acid (DLPA), and dipalmitoylphosphatidic acid (DPPA). Other suitable phospholipids include phosphatidylethanolamines, phosphatidylinositols, and phosphatidic acids containing lauric, myristic, stearoyl, and palmitic acid chains. Further, incorporation of polyethylene glycol (PEG) containing phospholipids is also contemplated by the present technology. It is contemplated by this technology to include cholesterol optionally in the liposomal formulation. Cholesterol is known to improve liposome stability and prevent loss of phospholipid to lipoproteins in vivo.

“Unilamellar liposomes,” also referred to as “single lamellar vesicles,” are spherical vesicles that include one lipid bilayer membrane that defines a single closed aqueous compartment. The bilayer membrane includes two layers (or “leaflets”) of lipids; an inner layer and an outer layer. The outer layer of the lipid molecules is oriented with the hydrophilic head portions toward the external aqueous environment and the hydrophobic tails pointed downward toward the interior of the liposome. The inner layer of the lipid lay directly beneath the outer layer with the lipids oriented with the heads facing the aqueous interior of the liposome and the tails oriented toward the tails of the outer layer of lipid.

“Multilamellar liposomes” also referred to as “multilamellar vesicles” or “multiple lamellar vesicles,” include more than one lipid bilayer membrane, which membranes define more than one closed aqueous compartment. The membranes are concentrically arranged so that the different membranes are separated by aqueous compartments, much like an onion.

The terms “bioactive agent” and “pharmaceutical agent” are used interchangeably and include but are not limited to, an antibiotic, an analgesic, an anesthetic, an antiacne agent, an antibiotic, an antibacterial, an anticancer agent, an anticholinergic, an anticoagulant, an antidyskinetic, an antiemetic, an antifibrotic, an antifungal, an antiglaucoma agent, an anti-inflammatory, an antineoplastic, an antiosteoporotic, an antipagetic, an anti-Parkinson's agent, an antisporatic, an antipyretic, an antiseptic, an antithrombotic, an antiviral, a calcium regulator, a keratolytic, and/or a sclerosing agent.

The terms “encapsulation” and “entrapped,” as used herein, refer to the incorporation or association of a biologically active (e.g., a pharmaceutical agent) in or with a liposome. The pharmaceutical agent may be associated with the lipid bilayer or present in the aqueous interior of the liposome, or both. In one embodiment, a portion of the encapsulated pharmaceutical agent takes the form of a precipitated salt in the interior of the liposome. The pharmaceutical agent may also self-precipitate in the interior of the liposome.

As used herein, “treat” or “treating” refers to: (i) preventing a pathologic condition (e.g., breast cancer; sepsis) from occurring (e.g. prophylaxis) or preventing symptoms related to the same; (ii) inhibiting the pathologic condition or arresting its development or inhibiting or arresting symptoms related to the same; or (iii) relieving the pathologic condition or relieving symptoms related to the same.

As used herein, the terms “subject” and “patient” refer to any animal, such as a mammal like a dog, cat, bird, livestock, and preferably a human.

As used herein, the term “effective amount” refers to the amount of a composition sufficient to effect beneficial or desired results. An effective amount can be administered in one or more administrations, applications, or dosages and is not intended to be limited to a particular formulation or administration route.

As used herein, the term “administration” refers to the act of giving a drug, prodrug, or other agent, or therapeutic treatment to a subject. Exemplary routes of administration to the human body can be through the eyes (ophthalmic), mouth (oral), skin (transdermal, topical), nose (nasal), lungs (inhalant), oral mucosa (buccal), ear, by injection (e.g., intravenously, subcutaneously, intratumorally, intraperitoneally, etc.), and the like.

As used herein, the term “pharmaceutical composition” refers to the combination of a biological agent with a carrier, inert or active, making the composition especially suitable for therapeutic use.

The terms “pharmaceutically acceptable” or “pharmacologically acceptable”, as used herein, refer to compositions that do not substantially produce adverse reactions, e.g., toxic, allergic, or immunological reactions, when administered to a subject.

As used herein, the term “treating” includes reducing or alleviating at least one adverse effect or symptom of a disease or disorder through introducing in any way a therapeutic composition of the present technology into or onto the body of a subject. “Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

As used herein, “therapeutically effective dose” refers to an amount of a therapeutic agent sufficient to bring about a beneficial or desired clinical effect. Said dose can be administered in one or more administrations. However, the precise determination of what would be considered an effective dose may be based on factors individual to each patient, including, but not limited to, the patient's age, size, type or extent of disease, stage of the disease, route of administration, the type or extent of supplemental therapy used, ongoing disease process, and type of treatment desired (e.g., aggressive versus conventional treatment).

### EMBODIMENTS OF THE TECHNOLOGY

1. Liposome Formation

The liposomes that are used in the present invention are formed from standard vesicle-forming lipids, which generally include neutral and negatively charged phospholipids and a sterol, such as cholesterol. The selection of lipids is generally guided by consideration of, e.g., liposome size and stability of the liposomes in the bloodstream.

Various types of lipids are used to produce liposomes. For example, amphipathic lipids that find use are zwitterionic, acidic, or cationic lipids. Examples of zwitterionic amphipathic lipids are phosphatidylcholines, phosphatidylethanolamines, sphingomyelins, etc. Examples of acidic amphipathic lipids are phosphatidylglycerols, phosphatidylserines, phosphatidylinositols, phosphatidic acids, etc. Examples of cationic amphipathic lipids are diacyl trimethylammonium propanes, diacyl dimethylammonium propanes, stearylamine, etc. Examples of neutral lipids include diglycerides, such as diolein, dipalmitolein, and mixed caprylin-caprin; triglycerides, such as triolein, tripalmitolein, trilinolein, tricaprylin, and trilaurin; and combinations thereof. Additionally, cholesterol or plant sterols are used in some embodiments, e.g., to make multivesicular liposomes.

In some embodiments, the major lipid component in the liposomes is phosphatidylcholine. Phosphatidylcholines having a variety of acyl chain groups of varying chain length and degree of saturation are available or may be isolated or synthesized by well-known techniques. In general, less saturated phosphatidylcholines are more easily sized, particularly when the liposomes must be sized below about 0.3 microns, e.g., for purposes of filter sterilization. In some embodiments, phosphatidylcholines containing saturated fatty acids with carbon chain lengths in the range of about C₁₄ to C₂₂ are preferred. Phosphatidylcholines with mono- or diunsaturated fatty acids and mixtures of saturated and unsaturated fatty acids are used in some embodiments. Other suitable lipids include phosphonolipids in which the fatty acids are linked to glycerol via ether linkages rather than ester linkages (e.g., as found in some members of the Archaea). Liposomes useful in the present invention may also be composed of sphingomyelin or phospholipids with head groups other than choline, such as ethanolamine, serine, glycerol, and inositol. In some embodiments, liposomes include a sterol, preferably cholesterol, at molar ratios of from 0.1 to 1.0 (cholesterol:phospholipid). In some embodiments, the liposome compositions are distearoylphosphatidylcholine/cholesterol, dipalmitoylphosphatidylcholine/cholesterol, and sphingomyelin/cholesterol. Methods used in sizing and filter-sterilizing liposomes are provided below.

A variety of methods are available for preparing liposomes as described in, e.g., Szoka et al., Ann. Rev. Biophys. Bioeng. 9:467 (1980), U.S. Pat. Nos. 4,235,871; 4,501,728; and 4,837,028; the text Liposomes, Marc J. Ostro, ed., Marcel Dekker, Inc., New York, 1983, Chapter 1, and Hope, et al., Chem. Phys. Lip. 40:89 (1986), all of which are incorporated herein by reference. One exemplary method produces multilamellar vesicles of heterogeneous sizes. In this method, the vesicle-forming lipids are dissolved in a suitable organic solvent or solvent system and dried under vacuum or an inert gas to form a thin lipid film. Alternatively, the lipids may be dissolved in a suitable solvent, such as tertiary butanol, and then lyophilized to form a more homogeneous lipid mixture that is in a more easily hydrated powder-like form. This film or powder is covered with an aqueous buffered solution and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate.

Many different types of organic solvents such as ethers, hydrocarbons, halogenated hydrocarbons, and/or Freons are used in some embodiments as the solvent in the lipid component. For example, diethyl ether, isopropyl ether, and other ethers; chloroform; tetrahydrofuran; halogenated ethers; esters, and combinations thereof find use in the present technology.

Several techniques are available for sizing liposomes to a desired size. One sizing method is described in U.S. Pat. No. 4,737,323, incorporated herein by reference. Sonicating a liposome suspension either by bath or probe sonication produces a progressive size reduction down to small unilamellar vesicles less than about 0.05 microns in size. Homogenization is another method that relies on shearing energy to fragment large liposomes into smaller ones. In a typical homogenization procedure, multilamellar vesicles are recirculated through a standard emulsion homogenizer until selected liposome sizes, typically between about 0.1 and 0.5 microns, are observed. In both methods, the particle size distribution can be monitored by conventional laser-beam particle size discrimination.

Extrusion of liposomes through a small-pore polycarbonate membrane or an asymmetric ceramic membrane is also an effective method for reducing liposome sizes to a relatively well-defined size distribution. Typically, the suspension is cycled through the membrane one or more times until the desired liposome size distribution is achieved. The liposomes may be extruded through successively smaller-pore membranes to achieve a gradual reduction in liposome size. For use in embodiments of the present technologies, liposomes having a size of from about 0.05 microns to about 0.15 microns are preferred.

As an example of one method for preparing liposomes, a process of preparing the formulation embodied in the present technology is initiated with the preparation of a solution from which the liposomes are formed. This is done, for example, by weighing out a quantity of a phosphatidylcholine, optionally cholesterol and/or optionally a phosphatidylglycerol, and dissolving them in an organic solvent, e.g., chloroform and methanol in a 1:1 mixture (v/v) or alternatively in neat chloroform. The solution is evaporated to form a solid lipid phase such as a film or a powder, for example, with a rotary evaporator, spray dryer, or other method. The film or powder is then hydrated with an aqueous solution optionally containing an excipient and having a pH range from about 2.0 to about 7.4 to form a liposome dispersion. The lipid film or powder dispersed in the aqueous solution is heated to a temperature from about 25° C. to about 70° C. depending on the phospholipids used.

Multilamellar liposomes are formed, e.g., by agitation of the dispersion, preferably through the use of a thin-film evaporator apparatus such as is described in U.S. Pat. No. 4,935,171 or through shaking or vortex mixing. Unilamellar vesicles are formed by the application of a shearing force to an aqueous dispersion of the lipid solid phase, e.g., by sonication or the use of a microfluidizing apparatus such as a homogenizer or a French press. Shearing force can also be applied using injection, freezing and thawing, dialyzing away a detergent solution from lipids, or other known methods used to prepare liposomes. The size of the liposomes can be controlled using a variety of known techniques including controlling the duration of shearing force. In some embodiments, a homogenizing apparatus is employed to produce unilamellar vesicles having diameters of less than 200 nanometers at a pressure of 3,000 to 14,000 psi (e.g., 10,000 to 14,000 psi) and a temperature that is about at the aggregate transition temperature of the lipids.

2. Bioactive Agents

In some embodiments, biological substances and/or therapeutic agents are incorporated by encapsulation within liposomes. Examples of bioactive agents include antianginal, antiarrhythmics, antiasthmatic agents, antibiotics, antidiabetics, antifungals, antihistamines, antihypertensives, antiparasitics, antineoplastics, antivirals, cardiac glycosides, herbicides, hormones, immunomodulators, monoclonal antibodies, neurotransmitters, nucleic acids, pesticides, proteins, radio contrast agents, radionuclides, sedatives, analgesics, steroids, tranquilizers, vaccines, vasopressors, anesthetics, and/or peptides.

The drugs that can be incorporated into the dispersion system as therapeutic agents include chemicals as well as biologics. The term “chemicals” encompasses compounds that are classically referred to as drugs, such as antitumor agents, anaesthetics, analgesics, antimicrobial agents, opiates, hormones, etc. Of particular interest for inclusion in the liposome compositions of the present invention are analgesics, e.g., opiates and/or opioids such as hydromorphone and buprenorphine.

The term “biologics” encompasses nucleic acids (e.g., DNA and RNA), proteins and peptides, and includes compounds such as cytokines, hormones (e.g., pituitary and hypophyseal hormones), growth factors, vaccines, etc.

Suitable antibiotics for inclusion in the liposome compositions of the present invention include, but are not limited to, loracarbef, cephalexin, cefadroxil, cefixime, ceftibuten, cefprozil, cefpodoxime, cephradine, cefuroxime, cefaclor, neomycin/polymyxin/bacitracin, dicloxacillin, nitrofurantoin, nitrofurantoin macrocrystal, nitrofurantoin/nitrofuran mac, dirithromycin, gemifloxacin, ampicillin, gatifloxacin, penicillin V potassium, ciprofloxacin, enoxacin, amoxicillin, amoxicillin/clavulanate potassium, clarithromycin, levofloxacin, moxifloxacin, azithromycin, sparfloxacin, cefflinir, ofloxacin, trovafloxacin, lomefloxacin, methenamine, erythromycin, norfloxacin, clindamycin/benzoyl peroxide, quinupristin/dalfopristin, doxycycline, amikacin sulfate, vancomycin, kanamycin, netilmicin, streptomycin, tobramycin sulfate, gentamicin sulfate, tetracyclines, framycetin, minocycline, nalidixic acid, demeclocycline, trimethoprim, miconazole, colistimethate, piperacillin sodium/tazobactam sodium, paromomycin, colistin/neomycin/hydrocortisone, amebicides, sulfisoxazole, pentamidine, sulfadiazine, clindamycin phosphate, metronidazole, oxacillin sodium, nafcillin sodium, vancomycin hydrochloride, clindamycin, cefotaxime sodium, co-trimoxazole, ticarcillin disodium, piperacillin sodium, ticarcillin disodium/clavulanate potassium, neomycin, daptomycin, cefazolin sodium, cefoxitin sodium, ceftizoxime sodium, penicillin G potassium and sodium, ceftriaxone sodium, ceftazidime, imipenem/cilastatin sodium, aztreonam, cinoxacin, erythromycin/sulfisoxazole, cefotetan disodium, ampicillin sodium/sulbactam sodium, cefoperazone sodium, cefamandole nafate, gentamicin, sulfisoxazole/phenazopyridine, tobramycin, lincomycin, neomycin/polymyxin B/gramicidin, clindamycin hydrochloride, lansoprazole/clarithromycin/amoxicillin, alatrofloxacin, linezolid, bismuth subsalicylate/metronidazole/tetracycline, erythromycin/benzoyl peroxide, mupirocin, fosfomycin, pentamidine isethionate, imipenem/cilastatin, troleandomycin, gatifloxacin, chloramphenicol, cycloserine, neomycin/polymyxin B/hydrocortisone, ertapenem, meropenem, cephalosporins, fluconazole, cefepime, sulfamethoxazole, sulfamethoxazole/trimethoprim, neomycin/polymyxin B, penicillins, rifampin/isoniazid, erythromycin estolate, erythromycin ethylsuccinate, erythromycin stearate, ampicillin trihydrate, ampicillin/probenecid, sulfasalazine, sulfanilamide, sodium sulfacetamide, dapsone, doxycycline hyclate, trimenthoprim/sulfa, methenamine mandelate, plasmodicides, pyrimethamine, hydroxychloroquine, chloroquine phosphate, trichomonocides, anthelmintics, atovaquone. In particularly preferred embodiments, doxycycline is loaded in the liposome compositions of the present invention.

3. Loading

The pharmaceutical agent is generally loaded into pre-formed liposomes using a loading procedure, for example, by pH gradient. In some embodiments, the loading begins by establishing an internal liposome pH of approximately pH 2 to 3. In some embodiments, the pharmaceutical agent may precipitate in the interior of the liposome. This precipitation protects the pharmaceutical agent and the lipids from degradation (e.g., hydrolysis). In some embodiments, an excipient such as citrate or sulfate precipitates the pharmaceutical agent and can be utilized in the interior of the liposomes together with a gradient to promote drug loading.

For example, according to embodiments of the technology, sulfuric acid is used to load liposomes. In some embodiments, liposomes, e.g., of DPPC/cholesterol, are loaded with a bioactive agent (e.g., an analgesic such as hydromorphone and/or buprenorphine). According to the technology, a lipid film is lyophilized from a solvent-treated (e.g., t-butanol-treated) lipid film comprising, e.g., DPPC and cholesterol in a defined ratio, e.g., at 2:1 DPPC:cholesterol. Then, liposomes are formed by adding sulfuric acid (e.g., at a concentration of 0.1 to 2.0 M, e.g., 0.375 M, 0.75 M, 1.5 M, etc.) and incubating, e.g., at 50° C. for 1 hour. In some embodiments, the acid solution external to the liposomes is neutralized with base, e.g., NaOH (e.g., 1 M) and then a bioactive agent (e.g., 20 mg of hydromorphone) is loaded into the liposomes, e.g., by incubating the drug with the liposomes for, e.g., 1 hour at 80° C. In some embodiments, liposomes are sedimented by centrifugation (and optionally washed and re-centrifuged one or more additional times) to remove unencapsulated biological agent.

4. Pharmaceutical Preparations

In some embodiments, the liposome compositions prepared by the methods described herein are administered alone or in a mixture with a physiologically-acceptable carrier (such as physiological saline or phosphate buffer) selected in accordance with the route of administration and standard pharmaceutical practice. Generally, normal saline is employed as the pharmaceutically acceptable carrier. Other suitable carriers include, e.g., water, buffered water, 0.4% saline, 0.3% glycine, and the like, including glycoproteins for enhanced stability, such as albumin, lipoprotein, globulin, etc. In compositions comprising saline or other salt-containing carriers, the carrier is preferably added following liposome formation. Thus, after the liposome is formed and loaded with a suitable drug, the liposome can be diluted into pharmaceutically acceptable carriers such as normal saline. These compositions may be sterilized by conventional, well known sterilization techniques. The resulting aqueous solutions may be packaged for use or filtered under aseptic conditions and lyophilized, the lyophilized preparation being combined with a sterile aqueous solution prior to administration. The compositions may also contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc. Additionally, the composition may include lipid-protective agents that protect lipids against free-radical and lipid-peroxidative damages on storage. Lipophilic free-radical quenchers, such as alpha-tocopherol and water-soluble iron-specific chelators, such as ferrioxamine, are suitable.

The concentration of liposomes in the pharmaceutical formulations can vary widely, e.g., from less than about 0.05%, usually at or at least about 2 to 5% to as much as 10 to 30% by weight and are selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, liposomes composed of irritating lipids may be diluted to low concentrations to lessen inflammation at the site of administration. The amount of liposomes administered will depend upon the particular drug used, the disease state being treated and the judgement of the clinician but will generally be between about 0.01 and about 50 mg per kilogram of body weight, preferably between about 0.1 and about 5 mg per kg of body weight.

In some embodiments, it is desirable to include polyethylene glycol (PEG)-modified phospholipids, PEG-ceramide, or ganglioside G_{M1}-modified lipids to the liposomes. Addition of such components prevents liposome aggregation and provides for increasing circulation lifetime and increasing the delivery of the loaded liposomes to the target tissues. Typically, the concentration of the PEG-modified phospholipids, PEG-ceramide, or G_{M1}-modified lipids in the liposome will be about 1 to 15%.

In some embodiments, overall liposome charge is an important determinant in liposome clearance from the blood. Charged liposomes are typically taken up more rapidly by the reticuloendothelial system (Juliano, Biochem. Biophys. Res. Commun. 63:651 (1975)) and thus have shorter half-lives in the bloodstream. Liposomes with prolonged circulation half-lives are typically desirable for therapeutic and certain diagnostic uses. For instance, liposomes that are maintained from 8, 12, or up to 24 hours in the bloodstream are particularly preferred.

In another example of their use, drug-loaded liposomes can be incorporated into a broad range of topical dosage forms including but not limited to gels, oils, emulsions, and the like. For instance, in some embodiments the suspension containing the drug-loaded liposomes is formulated and administered as a topical cream, paste, ointment, gel, lotion, and the like.

The present technology also provides liposome compositions in kit form. The kit will typically comprise a container that is compartmentalized for holding the various elements of the kit. The kit contains the compositions of the present inventions, preferably in dehydrated form, with instructions for their rehydration and administration.

In still other embodiments, the drug-loaded liposomes have a targeting moiety attached to the surface of the liposome. Methods of attaching targeting moieties (e.g., antibodies, proteins) to lipids (such as those used in the present particles) are known to those of skill in the art.

Dosage for the drug-loaded liposome formulations depends on the ratio of drug to lipid and the administrating physician's opinion based on age, weight, and condition of the patient.

In some embodiments, compositions comprising liposomes encapsulating a bioactive agent are formulated with a buffering agent. The buffering agent may be any pharmaceutically acceptable buffering agent. Buffer systems include citrate buffers, acetate buffers, borate buffers, and phosphate buffers. Examples of buffers include citric acid, sodium citrate, sodium acetate, acetic acid, sodium phosphate and phosphoric acid, sodium ascorbate, tartartic acid, maleic acid, glycine, sodium lactate, lactic acid, ascorbic acid, imidazole, sodium bicarbonate and carbonic acid, sodium succinate and succinic acid, histidine, and sodium benzoate and benzoic acid.

In some embodiments, compositions comprising liposomes encapsulating a bioactive agent are formulated with a chelating agent. The chelating agent may be any pharmaceutically acceptable chelating agent. Chelating agents include ethylenediaminetetraacetic acid (also synonymous with EDTA, edetic acid, versene acid, and sequestrene), and EDTA derivatives, such as dipotassium edetate, disodium edetate, edetate calcium disodium, sodium edetate, trisodium edetate, and potassium edetate. Other chelating agents include citric acid and derivatives thereof. Citric acid also is known as citric acid monohydrate. Derivatives of citric acid include anhydrous citric acid and trisodiumcitrate-dihydrate. Still other chelating agents include niacinamide and derivatives thereof and sodium desoxycholate and derivatives thereof.

In some embodiments, compositions comprising liposomes encapsulating a bioactive agent are formulated with an antioxidant. The antioxidant may be any pharmaceutically acceptable antioxidant. Antioxidants are well known to those of ordinary skill in the art and include materials such as ascorbic acid, ascorbic acid derivatives (e.g., ascorbylpalmitate, ascorbylstearate, sodium ascorbate, calcium ascorbate, etc.), butylated hydroxy anisole, butylated hydroxy toluene, alkylgallate, sodium meta-bisulfate, sodium bisulfate, sodium dithionite, sodium thioglycollic acid, sodium formaldehyde sulfoxylate, tocopherol and derivatives thereof, (d-alpha tocopherol, d-alpha tocopherol acetate, dl-alpha tocopherol acetate, d-alpha tocopherol succinate, beta tocopherol, delta tocopherol, gamma tocopherol, and d-alpha tocopherol polyoxyethylene glycol 1000 succinate) monothioglycerol, and sodium sulfite. Such materials are typically added in ranges from 0.01 to 2.0%.

In some embodiments, compositions comprising liposomes encapsulating a bioactive agent are formulated with a cryoprotectant. The cryoprotecting agent may be any pharmaceutically acceptable cryoprotecting agent. Common cryoprotecting agents include histidine, polyethylene glycol, polyvinyl pyrrolidine, lactose, sucrose, mannitol, and polyols.

In some embodiments, compositions comprising liposomes encapsulating a bioactive agent are formulated with an isotonicity agent. The isotonicity agent can be any pharmaceutically acceptable isotonicity agent. This term is used in the art interchangeably with iso-osmotic agent, and is known as a compound which is added to the pharmaceutical preparation to increase the osmotic pressure, e.g., in some embodiments to that of 0.9% sodium chloride solution, which is iso-osmotic with human extracellular fluids, such as plasma. Preferred isotonicity agents are sodium chloride, mannitol, sorbitol, lactose, dextrose and glycerol.

Compositions of the liposomes encapsulating a bioactive agent may optionally comprise a preservative. Common preservatives include those selected from the group consisting of chlorobutanol, parabens, thimerosol, benzyl alcohol, and phenol. Suitable preservatives include but are not limited to: chlorobutanol (0.30.9% W/V), parabens (0.01-5.0%), thimerosal (0.004-0.2%), benzyl alcohol (0.5-5%), phenol (0.1-1.0%), and the like.

In some embodiments, compositions comprising liposomes encapsulating a bioactive agent are formulated with a humectant to provide a pleasant mouth-feel in oral applications. Humectants known in the art include cholesterol, fatty acids, glycerin, lauric acid, magnesium stearate, pentaerythritol, and propylene glycol.

In some embodiments, an emulsifying agent is included in the formulations, for example, to ensure complete dissolution of all excipients, especially hydrophobic components such as benzyl alcohol. Many emulsifiers are known in the art, e.g., polysorbate 60.

For some embodiments related to oral administration, it may be desirable to add a pharmaceutically acceptable flavoring agent and/or sweetener. Compounds such as saccharin, glycerin, simple syrup, and sorbitol are useful as sweeteners.

5. Administration and Therapy

Once the therapeutic agent has been loaded into the liposomes, the combination can be administered to a patient by a variety of techniques.

Preferably, the pharmaceutical compositions are administered parenterally, e.g., intraarticularly, intravenously, intraperitoneally, subcutaneously, or intramuscularly. In some embodiments, the pharmaceutical compositions are administered intravenously or intraperitoneally by a bolus injection. For example, see Raham et al., U.S. Pat. No. 3,993,754; Sears, U.S. Pat. No. 4,145,410; Papahadjopoulos et al., U.S. Pat. No. 4,235,871; Schneider, U.S. Pat. No. 4,224,179; Lenk et al., U.S. Pat. No. 4,522,803; and Fountain et al., U.S. Pat. No. 4,588,578. Particular formulations that are suitable for this use are found in Remington's Pharmaceutical Sciences, Mack Publishing Company, Philadelphia, Pa., 17th ed. (1985). Typically, the formulations comprise a solution of the liposomes suspended in an acceptable carrier, preferably an aqueous carrier. A variety of aqueous carriers are used in embodiments of the technology, e.g., water, buffered water, 0.9% isotonic saline, and the like. These compositions may be sterilized by conventional, well known sterilization techniques, or may be sterile filtered. The resulting aqueous solutions may be packaged for use as is, or lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Dosage for the liposome formulations will depend on the ratio of drug to lipid and the administrating physician's opinion based on age, weight, and condition of the patient.

The methods of the present invention may be practiced in a variety of hosts. Preferred hosts include mammalian species, such as humans, non-human primates, dogs, cats, cattle, horses, sheep, and the like.

In other methods, the pharmaceutical preparations may be contacted with the target tissue by direct application of the preparation to the tissue. The application may be made by topical, “open”, or “closed” procedures. By “topical”, it is meant the direct application of the pharmaceutical preparation to a tissue exposed to the environment, such as the skin, oropharynx, external auditory canal, and the like. “Open” procedures are those procedures include incising the skin of a patient and directly visualizing the underlying tissue to which the pharmaceutical preparations are applied. This is generally accomplished by a surgical procedure, such as a thoracotomy to access the lungs, abdominal laparotomy to access abdominal viscera, or other direct surgical approach to the target tissue. “Closed” procedures are invasive procedures in which the internal target tissues are not directly visualized, but accessed via inserting instruments through small wounds in the skin. For example, the preparations may be administered to the peritoneum by needle lavage. Likewise, the pharmaceutical preparations may be administered to the meninges or spinal cord by infusion during a lumbar puncture followed by appropriate positioning of the patient as commonly practiced for spinal anesthesia or metrazamide imaging of the spinal cord. Alternatively, the preparations may be administered through endoscopic devices.

The compositions of the present invention that further comprise a targeting antibody on the surface of the liposome are particularly useful for the treatment of certain diseases.

The therapeutic use of liposomes can include the delivery of drugs that are normally toxic in the free form. In the liposomal form, the toxic drug may be directed away from the sensitive tissue where toxicity can result and targeted to selected areas where they can exert their therapeutic effects. Liposomes can also be used therapeutically to release drugs slowly, over a prolonged period of time, thereby reducing the frequency of drug administration through an enhanced pharmacokinetic profile. In addition, liposomes can provide a method for forming an aqueous dispersion of hydrophobic drugs for intravenous delivery.

The route of delivery of liposomes can also affect their distribution in the body. Passive delivery of liposomes involves the use of various routes of administration e.g., parenterally, although other effective administration forms, such as intraarticular injection, inhalant mists, orally active formulations, transdermal iotophoresis, or suppositories are also envisioned. Each route produces differences in localization of the liposomes.

Because dosage regimens for pharmaceutical agents are well known to medical practitioners, the amount of the liposomal pharmaceutical agent formulations that is effective or therapeutic for the treatment of a disease or condition in mammals and particularly in humans will be apparent to those skilled in the art. The optimal quantity and spacing of individual dosages of the formulations herein will be determined by the nature and extent of the condition being treated, the form, route and site of administration, and the particular patient being treated, and such optima can be determined by conventional techniques. It will also be appreciated by one of skill in the art that the optimal course of treatment, e.g., the number of doses given per day for a defined number of days, can be ascertained by those skilled in the art using conventional course of treatment determination tests.

The liposomes containing therapeutic agents and the pharmaceutical formulations thereof of the present technology and those produced by the processes thereof can be used therapeutically in animals (including man) in the treatment of infections or conditions which require: (1) repeated administrations, (2) the sustained delivery of the drug in its bioactive form, or (3) the decreased toxicity with suitable efficacy compared with the free drug in question.

The mode of administration of the liposomes containing the pharmaceutical agents and the pharmaceutical formulations thereof determine the sites and cells in the organism to which the compound will be delivered. The liposomes of the present technology can be administered alone but will generally be administered in admixture with a pharmaceutical carrier selected with regard to the intended route of administration and standard pharmaceutical practice. The preparations may be injected parenterally, for example, intravenously. For parenteral administration, they can be used, for example, in the form of a sterile aqueous solution which may contain other solutes, for example, enough salts or glucose to make the solution isotonic.

For the oral mode of administration, the liposomal therapeutic drug formulations of this technology can be used in the form of tablets, capsules; losenges, troches, powders, syrups, elixirs, aqueous solutions and suspensions, and the like. In the case of tablets, carriers which can be used include lactose, sodium citrate, and salts of phosphoric acid. Various disintegrants such as starch, and lubricating agents, such as magnesium stearate, sodium lauryl sulfate and talc, are commonly used in tablets. For oral administration in capsule form, useful diluents are lactose and high molecular weight polyethylene glycols. When aqueous suspensions are required for oral use, the active ingredient is combined with emulsifying and suspending agents. If desired, certain sweetening and/or flavoring agents can be added.

For the topical mode of administration, the liposomal therapeutic drug (e.g., antineoplastic drug) formulations of the present technology may be incorporated into dosage forms such as gels, oils, emulsions, and the like. Such preparations may be administered by direct application as a cream, paste, ointment, gel, lotion or the like.

For administration to humans in the curative, remissive, retardive, or prophylactic treatment of neoplastic diseases the prescribing physician will ultimately determine the appropriate dosage of the neoplastic drug for a given human subject, and this can be expected to vary according to the age, weight, and response of the individual as well as the nature and severity of the patient's disease. The dosage of the drug in liposomal form will generally be about that employed for the free drug. In some cases, however, it may be necessary to administer dosages outside these limits.

The term “therapeutically effective” as it pertains to the compositions of the invention means that a biologically active substance present in the aqueous component within the vesicles is released in a manner sufficient to achieve a particular medical effect for which the therapeutic agent is intended. Examples, without limitation, of desirable medical effects that can be attained are chemotherapy, antibiotic therapy, and regulation of metabolism. Exact dosages will vary depending upon such factors as the particular therapeutic agent and desirable medical effect, as well as patient factors such as age, sex, general condition, and the like. Those of skill in the art can readily take these factors into account and use them to establish effective therapeutic concentrations without resort to undue experimentation.

Generally, however, the dosage range appropriate for human use includes the range of 0.1 to 6000 mg/m² of body surface area. For some applications, such as subcutaneous administration, the dose required may be quite small, but for other applications, such as intraperitoneal administration, the dose desired to be used may be very large. While doses outside the foregoing dose range may be given, this range encompasses the breadth of use for practically all the biologically active substances.

The liposomes may be administered for therapeutic applications by any desired route, for example, intramuscular, intraarticular, epidural, intrathecal, intraperitoneal, subcutaneous, intravenous, intralymphatic, oral and submucosal, and by implantation under many different kinds of epithelia, including the bronchialar epithelia, the gastrointestinal epithelia, the urogenital epithelia, and various mucous membranes of the body.

In addition, the liposomes of the invention can be used to encapsulate compounds useful in agricultural applications, such as fertilizers, pesticides, and the like. For use in agriculture, the liposomes can be sprayed or spread onto an area of soil where plants will grow and the agriculturally effective compound contained in the vesicles will be released at a controlled rate by contact with rain and irrigation waters. Alternatively the slow-releasing vesicles can be mixed into irrigation waters to be applied to plants and crops. One skilled in the art will be able to select an effective amount of the compound useful in agricultural applications to accomplish the particular goal desired, such as the killing of pests, the nurture of plants, etc.

Although the disclosure herein refers to certain illustrated embodiments, it is to be understood that these embodiments are presented by way of example and not by way of limitation.

### EXAMPLES

### Materials and Methods

Liposomes were prepared from a mixture of dipalmitoylphosphatidylcholine (DPPC) and cholesterol by first mixing 80 micromol of DPPC (60 mg) and 40 micromol of cholesterol in chloroform. Chloroform was removed by flash evaporation and the lipid mixture was dissolved in 1 ml of warm tert-butanol (also referred to as t-butanol). The tert-butanol solution of lipid was then frozen rapidly by immersing the tube in a mixture of isopropanol and dry ice. After freezing, the mixture was lyophilized for 24 to 48 hours to produce a microporous mass of lipid ready for hydration. The lipid was hydrated by adding 1 ml of sulfuric acid solution (0.1875 to 1.5 M). The mixture was shaken at 50° C. for 1.5 hours. Once the lipid was hydrated, 20 mg of hydromorphone hydrochloride was added to the solution and the solution was shaken gently to dissolve. The excess sulfuric acid was then neutralized by adding an appropriate volume of 5 M sodium hydroxide alone or with phosphate buffer. The mixture was incubated for a further 1.5 hours at 50-85° C. to allow for complete drug loading. After drug loading, the mixture was diluted with 0.9% w/v sodium chloride and sedimented in an ultracentrifuge for 1 hour at 30,000×g. The supernatant was carefully removed and the pellet was re-suspended in a small volume of 0.9% w/v NaCl.

### Example 1

During the development of embodiments of the technology provided herein, experiments were performed to test loading of hydromorphone into DPPC liposomes using sulfuric acid loading. First, DPPC/cholesterol films were lyophilized from t-butanol-treated lipid films. Liposomes were formed in 0.375 M H₂SO₄ for 1 hour at 50° C. The acid solution was neutralized using NaOH (e.g., at 5 or 1.67 M), with and without phosphate buffer (e.g., at 0.11 M), and 20 mg of hydromorphone was added to the solution. The drug was loaded at 80° C. for 1.5 hours. Liposomes were sedimented by centrifugation at 30,000 rpm for 0.5 hour to remove any unencapsulated drug.

To measure the encapsulated contents of the liposomes, the liposomes were washed in 0.9% NaCl solution and solubilized with 1:3:1 NaCl:methanol:chloroform. Drug concentration was determined using spectrophotometry (Table 1).

TABLE 1
Capture efficiency of hydromorphone loading

| | Drug Loading |
|---|---|
| Lipid Composition | (% of Added) |
| Hydromorphone | 35 |
| DPPC:Cholesterol | |
| 2:1 | |
| Ammonium sulfate gradient | |
| 240 mM | |
| Hydromorphone | 56 |
| DPPC:Cholesterol | |
| 2:1 | |
| 0.375M Sulfuric Acid | |
| Unbuffered system | |
| Hydromorphone | 62 |
| DPPC:Cholesterol | |
| 2:1 | |
| 0.375M Sulfuric Acid | |
| Phosphate buffered loading | |

In additional experiments with buprenorphine chloroquine, data were collected demonstrating that buprenorphine and chloroquine are also efficiently loaded 100% using the acid loading methodology (Table 2). Experiments tested acid loading of liposomes at a sulfuric acid concentration of 0.375 M. A mass of 6 mg of buprenorphine and 33 mg of chloroquine were added to the compositions for loading into the liposomes.

TABLE 2
Capture Efficiency of buprenorphine and chloroquine loading

| | Drug Loading |
|---|---|
| Lipid Composition | (% of Added) |
| DPPC:Cholesterol | 69.9 |
| 2:1 | |
| 0.375M Sulfuric Acid | |
| 6 mg buprenorphine added | |
| Unbuffered system | |
| DPPC:Cholesterol | 100 |
| 2:1 | |
| 0.375M Sulfuric Acid | |
| 6 mg buprenorphine added | |
| Phosphate buffered loading | |
| DPPC:Cholesterol | 100 |
| 2:1 | |
| 0.375M Sulfuric Acid | |
| 33 mg chloroquine added | |
| Phosphate buffered loading | |

### Example 2

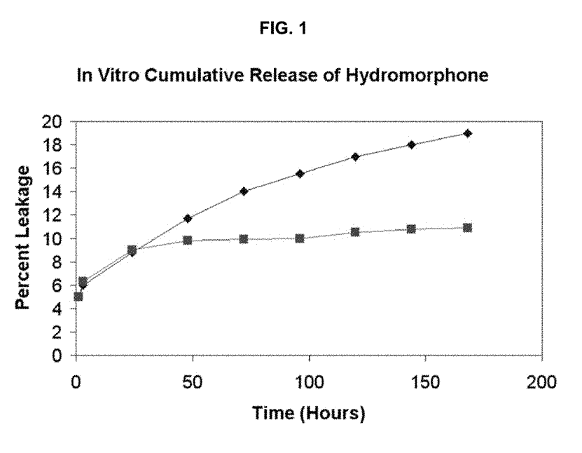

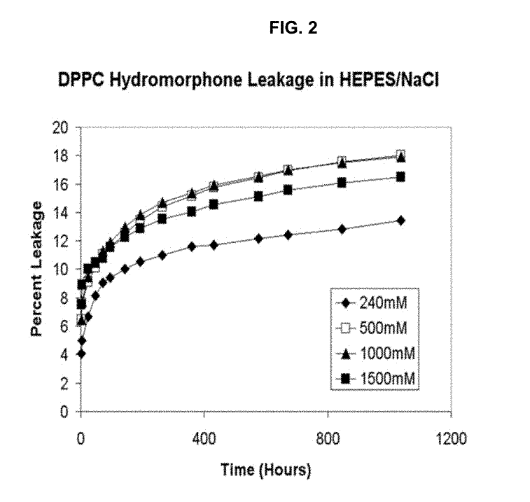

During the development of embodiments of the technology provided, experiments were performed to measure the leakage of drug from the loaded liposomes. Leakage studies were performed using the three preparations made by acid loading in Example 1 and shown in Table 1. Dialysis tubing was tied at one end. A volume of 0.5 mL of liposome preparation and 0.5 mL of physiologic saline solution were added to the dialysis tubing and the tubing was tied at the opposite end. The dialysis bag was suspended in 10 mL of saline solution in a 50 mL centrifuge tube. Unbuffered saline was used in this experiment because previous experiments demonstrated that unbuffered saline produced the fastest release from liposomal preparations and thus unbuffered saline provided a “worst case” scenario for liposome leakage. Accordingly, a liposome preparation that leaked slowly in unbuffered saline would be expected to leak even more slowly in a buffered medium such as HEPES and in the highly buffered environment in vivo. The tubes were covered with foil and agitated at 22° C. Aliquots of the saline solution were assayed for absorbance at 282 nm, the peak absorbance of hydromorphone. All of the acid loaded liposome preparations had release times of less than 20% over 72 hours. Nearly all of the hydromorphone release occurred within 24 hours (FIG. 1). Similarly, the two best acid loaded formulations of buprenorphine leaked less than 10% over 96 hours (FIG. 2).

### Example 3

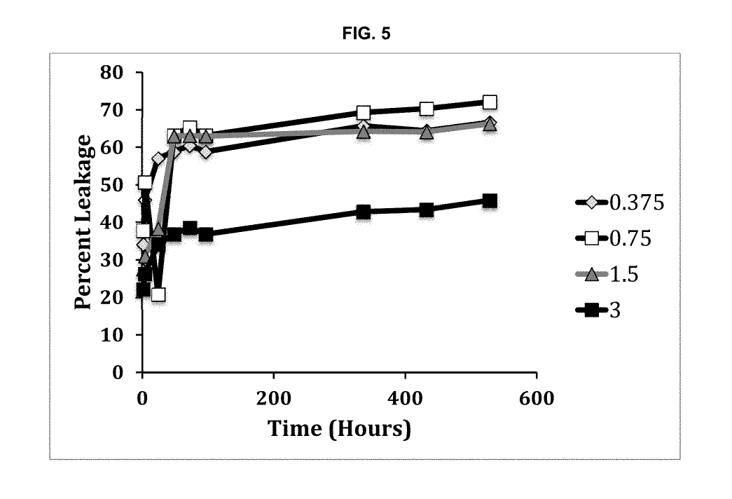

DPPC/cholesterol lipid masses (60/20 μM) were swelled in varying concentrations of sulfuric acid at 50° C. for 30 min. The swelled lipid mass was divided into 4 glass test tubes, and over laid with 8.25 mg of doxycycline hyclate in 1 mL of sterile water. A pH gradient between the inner compartment of the liposome and the outer solution was established by adding 5 M NaOH in 1 M citrate buffer (200, 400, 800 and 1600 μL for H2SO4 concentrations of 0.375, 0.75, 1.5 and 3.0 M respectively). Doxycycline was loaded at 22° C. for 24 hrs on a laboratory shaker. Liposomes were sedimented by centrifugation and resuspended in sterile physiologic saline. Liposomal doxycycline preparations were quantitated by spectrophotometry at a peak wave length of 245 nM. An aliquot of 20 μL of each preparation was placed in a dialysis bag. The bag was filled with 980 μL of sterile saline, and the bag was closed with an overhand knot. The dialysis bags were placed in 9 mL of sterile physiologic saline in 50 mL centrifuge tubes. The tubes were placed on a laboratory shaker and serial samples of the saline in the tube was removed, placed in a cuvette and analyzed spectophotometrically at 245 nM. The results are shown in FIG. 5.

### Example 4

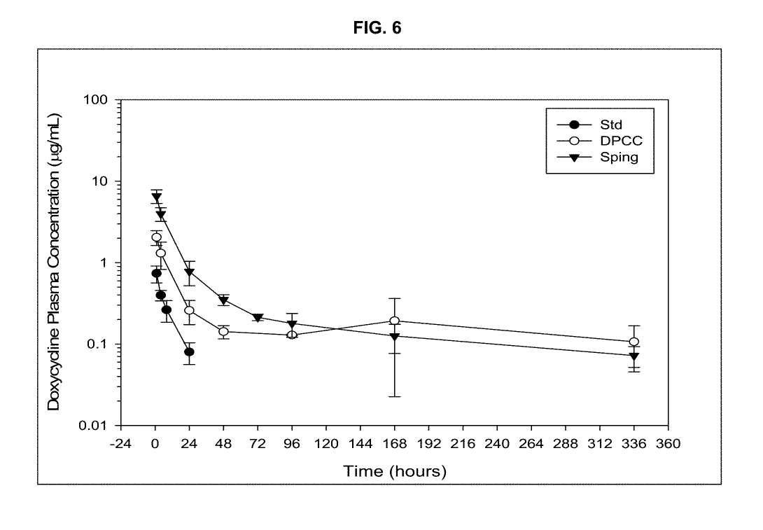

Groups of male and female ACI rats (n=4-5/group) were administered either the standard pharmaceutical formulation of doxycycline hyclate (Std), liposomal doxycycline in dipalmitoylphosphatidylcholine/cholesterol shell (DPCC) or liposomal doxycycline in egg sphingomyelin/cholesterol shells (Sping). Liposomal formulations were made using acid-loading technology as described in Example 3. Blood samples were drawn from the tail artery at serial time points after administration. Serum was separated from formed elements by centrifugation. Serum was frozen at −20° C. until analysis by HPLC. The results are shown in FIG. 6.

### Example 5

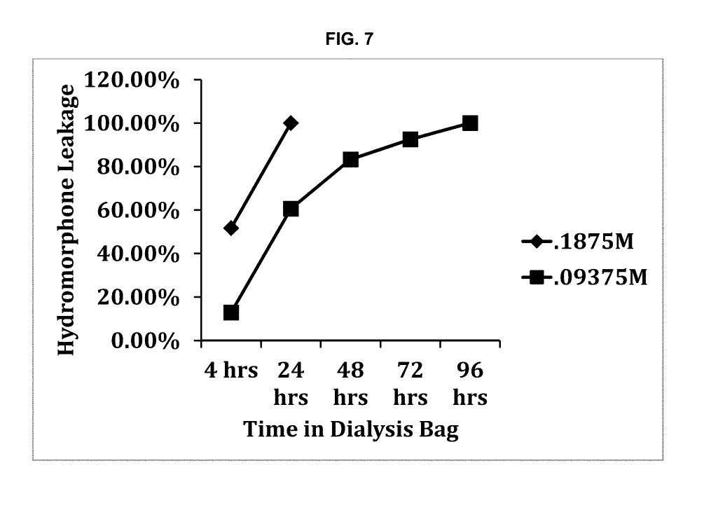

DPPC/cholesterol lipid masses (60/20 μM) were swelled in two concentrations of nitric acid at 50° C. for 30 min. 40 mg of hydromorphone powder was added to the swelled lipid mass in volume of 1 mL. 5 M NaOH was added to neutralize the solution outside the liposome and create a pH gradient between the inner and outer liposomal compartments. Hydromorphone was loaded at 55° C. for 1 hr in a water bath. Liposomes were sedimented by centrifugation and resuspended in sterile physiologic saline. Hydromorphone preparations were quantitated using spectrophotometry as described above for doxycycline, except that the peak wave length used was 282 nM. Leakage studies were performed as described for doxycycline, except that 100 μL of the liposomal hydromorphone and 900 μL of sterile saline were placed in the dialysis bag. The results are shown in FIG. 7.

### Example 6

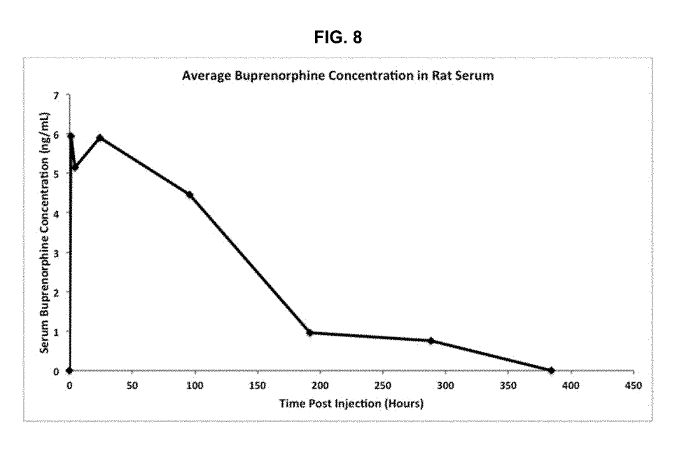

For preliminary pharmacokinetics experiments, 5 male Sprague Dawley rats were administered a single subcutaneous dose of liposomal buprenorphine at 3 mg/kg. Blood samples were drawn prior to injection and at the indicated time points after injection. The serum was separated from the formed elements by centrifugation, collected and stored at −70° C. and assayed using a commercially-available ELISA assay (Neogen Corp.) (FIG. 8).

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the technology as claimed should not be unduly limited to such specific embodiments. Indeed, various modifications of the described modes for carrying out the technology that are obvious to those skilled in pharmacology, biochemistry, medical science, or related fields are intended to be within the scope of the following claims.

We claim:

1. A composition comprising liposomes, sulfate ions, and hydrogen ions, wherein the concentration of the hydrogen ions inside the liposomes is greater than the concentration of the hydrogen ions outside the liposomes.

2. The composition of claim 1 comprising sulfuric acid.

3. The composition of claim 1, wherein the interior of said liposomes has a pH of at least 3 pH units lower than the exterior of said liposomes.

4. The composition of claim 1 comprising a bioactive agent in the interior of the liposomes.

5. The composition of claim 4 wherein said bioactive agent is an analgesic.

6. The composition of claim 4 wherein said bioactive agent is an opioid.

7. The composition of claim 4 wherein said bioactive agent is selected from the group consisting of hydromorphone, chloroquine, and buprenorphine.

8. The composition of claim 4 wherein said bioactive agent is an antibiotic.

9. The composition of claim 8, wherein said antibiotic is doxycycline.

10. The composition of claim 4 wherein the bioactive agent is selected from the group consisting of an antitumor agent, an anaesthetic, an analgesic, an antimicrobial agent, a hormone, an antiasthmatic agent, a cardiac glycoside, an antihypertensive, a vaccine, an antiarrhythmic, an immunomodulator, a steroid, a monoclonal antibody, a neurotransmitter, a radionuclide, a radio contrast agent, a nucleic acid, a protein, a herbicide, a pesticide, and suitable combinations thereof.

11. The composition of claim 1 comprising an aqueous buffer and a base outside the liposomes.

12. The composition of claim 1 comprising a sodium salt of an acid outside the liposomes.

13. The composition of claim 1 wherein the liposomes comprise phosphatidylcholine.

14. The composition of claim 1 wherein the liposomes comprise:
a) a phosphatidylcholine selected from the group consisting of distearoylphosphatidylcholine, hydrogenated soy phosphatidylcholine, hydrogenated egg phosphatidylcholine, dipalmitoylphosphatidylcholine, dimyristoylphosphatidylcholine, and dielaidoylphosphatidylcholine;
b) a sphingomyelin;
c) a neutral lipid; or
d) an acidic phospholipid.

15. A method for preparing liposomes encapsulating a bioactive agent, the method comprising:
1) forming liposomes having a concentration of sulfate ions inside the liposomes and further having a concentration of hydrogen ions inside the liposomes that is greater than the concentration of the hydrogen ions outside the liposomes; and
2) loading the liposomes with a bioactive agent by incubating the liposomes with the bioactive agent.

16. The method of claim 15, wherein the interior of said liposomes has a pH of at least 3 pH units lower than the exterior of said liposomes.

17. The method of claim 15, wherein forming the liposomes comprises forming liposomes in the presence of sulfuric acid.

18. The method of claim 15, further comprising adding a base to increase the pH outside the liposomes.

19. The method of claim 15, wherein the bioactive agent is an antibiotic.

20. A method of treating a subject in need of pain reduction, the method comprising:
1) administering to the subject a composition according to claim 5; and
2) assessing the subject's pain.
